(19) United States
(12) Patent Application Publication
(10) Pub. No.: US 2026/0013937 A1
(43) Pub. Date: Jan. 15, 2026

(54) TEMPERATURE CONTROL TO PREVENT SKIN BURN DURING MICROWAVE ABLATION PROCEDURE

(71) Applicant: Neuwave Medical, Inc., Madison, WI (US)

(72) Inventors: Jonathan DUFF, Madison, WI (US); Jason BRUNKOW, Mcfarland, WI (US); Mark THOM, Verona, WI (US); Christopher UNSETH, Sun Prairie, WI (US); Emily FORAN, Sun Prairie, WI (US)

(73) Assignee: Neuwave Medical, Inc., (US)

(21) Appl. No.: 18/767,748

(22) Filed: Jul. 9, 2024

(51) Int. Cl. A61B 18/18; A61B 17/00; A61B 18/00

(52) U.S. Cl. CPC A61B 18/1815; A61B 2017/00199; A61B 2017/00862; A61B 2018/00023; A61B 2018/00577; A61B 2018/00642; A61B 2018/00714; A61B 2018/00744; A61B 2018/00791; A61B 2018/1869

(57) ABSTRACT

An energy delivery device is disclosed including a cannula extendable through skin of a patient, an antenna extending from the cannula and operable to deliver energy to tissue of the patient, and a temperature regulator movable along a length of the cannula and operable to control a temperature of the skin as the antenna provides energy to the tissue.

### BACKGROUND

Ablation is an important therapeutic strategy for treating certain tissues, such as benign and malignant tumors, cardiac arrhythmias, cardiac dysrhythmias, and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

One challenge associated with microwave ablation is the potential for skin burn, such as at or near the point of entry of the microwave ablation device and shaft. Skin burns are a critical concern in microwave ablation procedures as they can lead to patient discomfort, prolonged recovery times, and could necessitate additional medical interventions. The risk of skin burn arises from the deposition of microwave energy in tissue that is close to the skin surface. Such localized heating can exceed a threshold for tissue damage, resulting in thermal injury to the skin and the underlying structures.

Accordingly, there is a need for improved systems and devices for preventing skin burns during microwave ablation procedures.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system that may be used in accordance with the principles of the present disclosure.

FIG. 2 is a schematic diagram of an example energy delivery device that may be used in accordance with the principles of the present disclosure.

FIG. 3 is the energy delivery device of FIG. 2 including a thermal regulator, according to at least one aspect of the present disclosure.

FIG. 4 is the thermal regulator of FIG. 3 pushed distally against the skin of a patient, according to at least one aspect of the present disclosure.

FIG. 5 is the energy delivery device of FIG. 2 including an alternative thermal regulator, according to one or more additional aspects of the present disclosure.

### DETAILED DESCRIPTION

The present disclosure is related to systems and methods for delivering energy to tissue for ablation operation and, more particularly, to systems and methods for controlling the temperature of the skin to prevent burns thereto during an energy delivery procedure.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

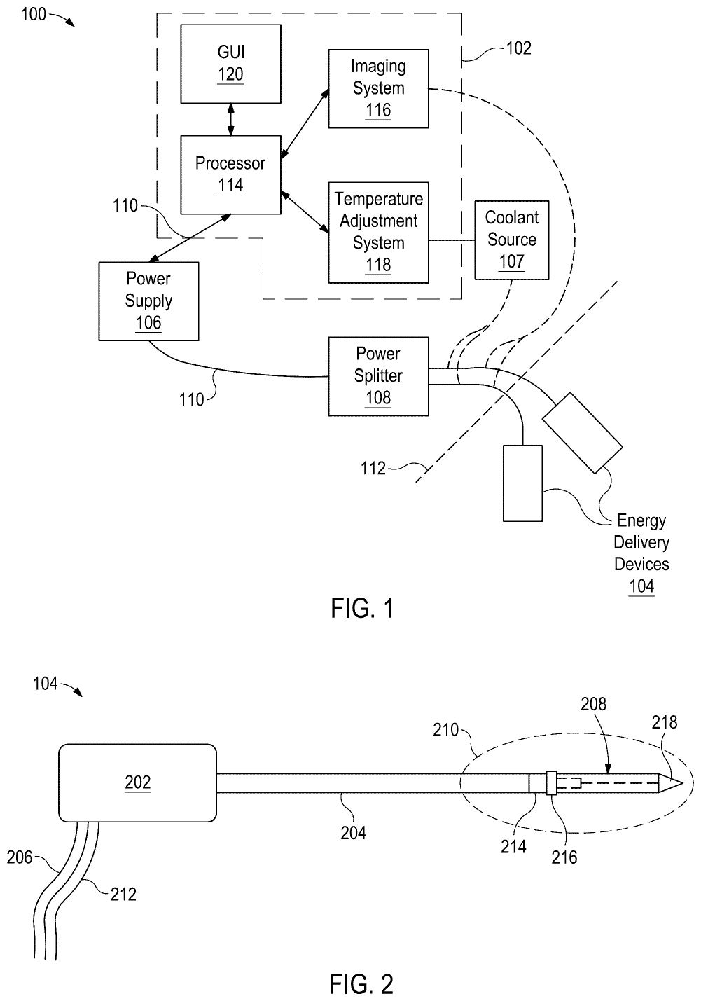

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. The system 100 further includes a power supply 106 communicably coupled to the control system 102 and the ablation probes 104 to provide electrical power thereto. In some applications, the system 100 may further include a power splitter 108 interposing the ablation probes 104 and the power supply 106 and configured to direct, control, and deliver power to the ablation probes 104. The system 100 further includes a coolant supply 107 communicably coupled to the control system 102 and the ablation probes 104 to provide coolant fluid thereto.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The control system 102 is configured to monitor, control, and provide feedback concerning operation of the system 100. As illustrated, the control system 102 includes at least a processor 114, an imaging system 116, and a temperature adjustment system 118. The control system 102 may further include a graphical user interface (GUI) 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The processor 114 may be provided within a computer system or module, which may include software or software instructions executable by the processor 114 to carry out functions and operations of the system 100. The software may be stored on a computer memory or computer memory device comprising any storage media readable by the processor 114. Examples of the computer memory include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 114. Examples of computer readable media include, but are not limited to, optical discs, magnetic disks, magnetic tape, solid-state media, and servers for streaming media over networks.

Based on instructions provided by the software, the processor 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The processor 114 interacts with the ablation probes 104 to raise or lower (e.g., tune) the amount of energy delivered to the tissue region. The processor 114 may also be configured to prime coolants for distribution into the ablation probes 104 such that the coolant is delivered at a desired temperature.

In some applications, the type of tissue being treated is inputted into the software for purposes of allowing the processor 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected ablation sizes may be based on tissue type. In such embodiments, the processor 114 may not control power delivery based on tissue type. In yet other embodiments, the processor 114 generates a chart or diagram based upon a particular type of tissue or tissue region displaying characteristics useful to a user of the system.

The processor 114 may allow a user to choose power, duration of treatment, different treatment algorithms for different tissue types, simultaneous application of power to multiple probes 104, coherent and incoherent phasing, etc. The processor 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

FIG. 2 is a schematic diagram of a probe 104 that may be used in accordance with the principles of the present disclosure, according to one or more embodiments. As indicated above, the ablation probe 104 may be configured to deliver (emit) energy (e.g., microwave energy, radiofrequency energy, radiation energy) to a target tissue region. As illustrated, the ablation probe 104 includes a handle housing 202 and an elongate shaft or probe cannula 204 extending distally from the handle 202.

A cable or cable assembly 206 may be operatively coupled to the handle 202 and configured to convey electrical power thereto. The cable assembly 206 may extend from the power supply 106 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210. The material of the antenna 208 is durable and provides a high dielectric constant. In some applications, the material of the antenna 208 is zirconium and/or a functional equivalent of zirconium. In at least one application, the ablation probe 104 includes two or more separate antennae 208 attached to the same or different power supplies.

In some applications, a cooling tube 212 is operatively coupled and configured to convey a cooling fluid or “coolant” to the handle 202. The handle 202 may be configured to control conveyance of the cooling fluid into and out of the probe cannula 204 to help regulate a temperature of the antenna 208 and the ablation zone 210. Example cooling fluids include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some applications, the ablation probe 104 may include a stick region 214, alternately referred to as a “tissue-loc” region, provided on the probe cannula at or near the antenna 208. The stick region 214 is designed to attain and maintain a temperature that accommodates adherence of a tissue region onto its surface. More specifically, the stick region 214 may operate as an anchoring element having a circulating agent or “coolant” (e.g., a gas delivered at or near its critical point; CO2) that freezes the interface between the stick region 214 and the adjacent tissue, thereby sticking (maintaining, locking, etc.) the antenna 208 in place during operation. The coolant may be provided to the stick region 214 via the cooling tube 212 from the coolant source 107, for example. Once a pre-determined low temperature is reached at the stick region 214, contact with adjacent tissue causes the tissue to adhere to the stick region 214, thereby resulting in attachment of the energy delivery device 204 to the tissue. During ablation, as the tissue warms, the antenna 208 remains secured to the tissue region due to tissue desiccation and charring. The stick region 214 may be made of any material able to attain and maintain a temperature such that upon contact with tissue induces adherence of the tissue onto the stick region 214. Example materials for the stick region 214 include, but are not limited to, a metal.

In some applications, the ablation probe 104 may further include a plug region 216 provided on the probe cannula 204 at or near the antenna 208. In at least one application, as depicted, the plug region 216 may be provided distal to the stick region 214 and otherwise interposing the stick region 214 and the antenna 208. The plug region 216 may be configured to prevent a reduction in temperature resulting from the cooled probe cannula 204 and the stick region 214 from affecting (e.g., reducing) the temperature within the antenna 208. Accordingly, the plug region 216 separates interior portions of the ablation probe 104 to prevent cooling or heating of a portion or portions of the device 104 while permitting cooling or heating of other portions. The plug region 216 may be made of an insulative material capable of being in contact with a material or region having a low temperature without having its temperature significantly reduced. Example insulative materials for the plug region 216 include, but are not limited to, a synthetic polymer (e.g., polystyrene, polyicynene, polyurethane, polyisocyanurate), aerogel, fiberglass, cork, or any combination thereof.

In some applications, the ablation probe 104 may further include a sharp stylet tip or “stylet” 218 positioned at the distal end of the antenna 208 and otherwise forming the distal end of the ablation probe 104. When included, the stylet 218 is designed to facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be braised to zirconia or an equivalent of zirconia. In such applications, the stylet 218 may comprise an extension of a metal portion of the antenna 208 and may be electrically active.

In some applications, the ablation probe 104 may have a coaxial transmission line positioned within the antenna 208, and a coaxial transmission line connecting with the antenna 208. In other embodiments, the ablation probe 104 may comprise a triaxial microwave probe with optimized tuning capabilities. The ablation probe 104 may be the same as or similar to any of the energy delivery devices described in U.S. Pat. No. 11,638,607, entitled “ENERGY DELIVERY SYSTEMS AND USES THEREOF”, which issued on May 2, 2023, the contents of which are hereby incorporated by reference in their entirety herein.

Referring again to FIG. 1, the control system 102 further includes the imaging system 116, which is in communication with the processor 114 and comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the system 100 provides software configured for use with imaging equipment of the imaging system 116, such as CT, MRI, and ultrasound, and generate two-dimensional (2D) and or three-dimensional (3D) images viewable by a user on the GUI 120. In some embodiments, the imaging equipment software allows a user to make predictions based upon known thermodynamic and electrical properties of tissue, vasculature, and location of the antenna(s) 208 (FIG. 2). In some embodiments, the imaging software allows the generation of a 2D or 3D map of the location of a tissue region (e.g., tumor, arrhythmia), location of the antenna(s) 208, and to generate a predicted map of the ablation zone 210 (FIG. 2).

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The power supply 106 may be configured to supply the energy required to operate the system 100. The power supply 106 is also configured to supply energy to the ablation probes 104, such as microwave energy, radiofrequency energy, radiation, cryo energy, electroporation, high intensity focused ultrasound, or any combination thereof. In accordance with principles of the present disclosure, the power supply 106 supplies microwave energy to the ablation probes 104 for purposes of tissue ablation. More specifically, power may be supplied to the ablation probes 102, but the microwave energy is generated in a microwave generator and sent to the antenna 208. In some applications, the power supply 106 may include one or more energy generators configured to provide as much as 100 watts of microwave power of a frequency of from 915 MHz to 5.8 GHZ, although the present invention is not so limited. The power splitter 108 may comprise a power distribution system operable to distribute the energy from the power supply 106 to the ablation probes 104. The power splitter 108 may be configured to provide varying energy levels to different regions of the ablation probes 104.

The temperature adjustment system 118 may be configured to use coolant systems (like the coolant source 107) and cooling fluids to help reduce undesired heating within and along the ablation probes 104. In particular, the temperature adjustment system 118 may include the coolant source 107 (FIG. 1) and the cooling tube 212 (FIG. 2) and may communicate with the handle 202 (FIG. 2) of each probe 104 to control conveyance of the cooling fluid into and out of the probe cannula 204 (FIG. 2), and thereby help regulate a temperature of the antenna 208 (FIG. 2) and the ablation zone 210 (FIG. 2). In some applications, the temperature adjustment system 118 may also be configured to communicate with the handle 202 to operate the stick region 214 and thereby attain and maintain a temperature that accommodates adherence of tissue onto its surface. For instance, in some embodiments, a user provides an input to an input interface, such as the GUI 120. Based on the user input, the temperature adjustment system 118 can control the coolant source 107 to provide coolant to the stick region 214, thereby adhering the stick region 214 to the adjacent tissue.

The temperature adjustment system 118 may also be configured to continuously or intermittently monitor the real-time temperature of the ablation probes 104. In such embodiments, the temperature adjustment system 118 may communicate with one or more temperature sensors (e.g., thermocouples) terminating at various points along the probe cannula 204 (FIG. 2) and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length. In some embodiments, the probe cannula 204 includes a first temperature sensor, a second temperature sensor, and a third temperature sensor. The first temperature sensor is placed at, or slightly proximal to, the antenna 208 to provide real-temperature measurements of the tissue being heated by the antenna 208. The second temperature sensor is placed at, or adjacent to, the stick region 214 to provide real-time temperature measurements of the tissue that is being cooled, and thus adhered to, the stick region 214. The third temperature sensor is proximal to the first and second temperature sensors along the cannula 204, such as at the point of entry into the skin, to provide real-time measurements of the patent's skin. The control system 102 can receive the temperature measurements from the first, second, and third sensors to control the coolant systems and cooling fluids from the temperature adjustment system 118 to the stick region 214 and/or other cooling systems of the energy delivery device 104, as will be described in more detail below.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the processor 114 to provide real-time temperature information to a user and display such measurements on the GUI 120. In at least one embodiment, based on the temperature data obtained by the temperature adjustment system 118, the processor 114 may be configured to autonomously adjust operation of the system 100 appropriately.

As described above, one challenge associated with microwave ablation is the potential for skin burn at or near the point of entry of the microwave ablation device. Current temperature adjustment systems may not provide adequate temperature regulation to stop or prevent skin burns when the antenna 208 is positioned close to the skin. According to embodiments of the present disclosure, a secondary cooling system may be included and operable to provide additional cooling capabilities to regulate the temperature of the skin, and thus, prevent, or at least substantially reduce, skin burn during microwave ablation procedures.

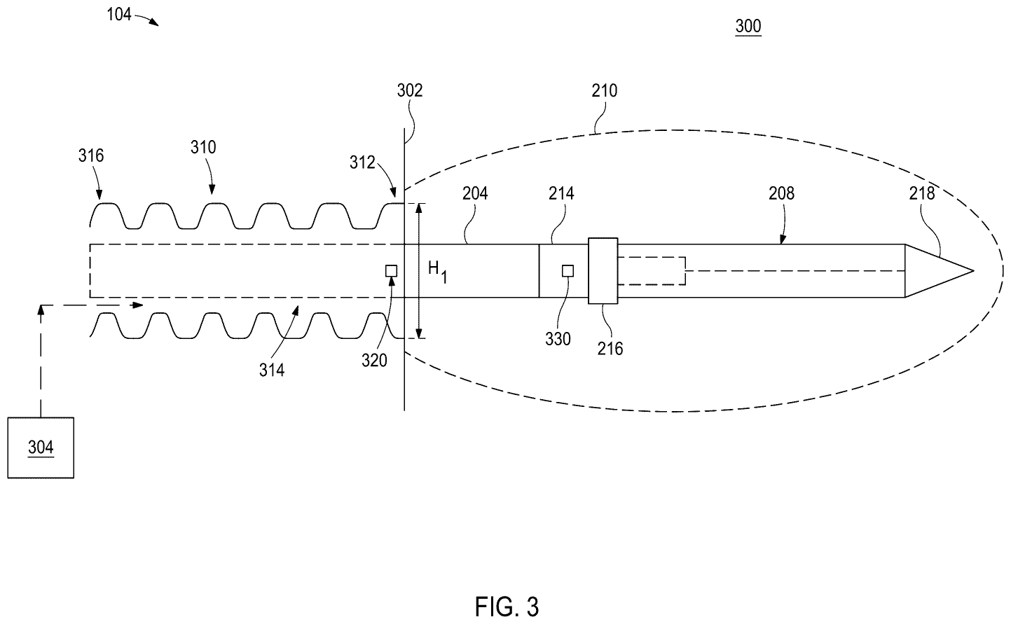

FIG. 3 is an enlarged view of a portion of the energy delivery device 104 of FIG. 2, according to one or more embodiments. As illustrated, the energy delivery device 104 is positioned such that the antenna 208 is percutaneously inserted into a patient 300 and positioned near the patient's skin 302. In operation, as described above, the stick region 214 of the energy delivery device 104 is provided with coolant to anchor the energy delivery device 104 to tissue positioned adjacent thereto. Once satisfied with the placement of the energy delivery device 104, the antenna 208 is provided with electrical power to emit energy to a target tissue region and thereby generate the ablation zone 210.

In some instances, as shown in FIG. 3, the proximal end of the ablation zone 210 may intersect or overlap the patient's skin 302, which may cause thermal damage to the skin 302. While some of the thermal energy spread is controlled by reduced temperature at the stick region 214, the stick region 214 alone is often not adequate in cooling the skin 302, which may result in the skin 302 being positioned within the ablation zone 210. To help regulate the temperature of the skin 302 due to the effects of ablation that occurs near the skin 302, the system 100 (FIG. 1) and the energy delivery device 104 may further include a secondary cooling system in the form of a temperature regulated flexible channel or “temperature regulator” 310.

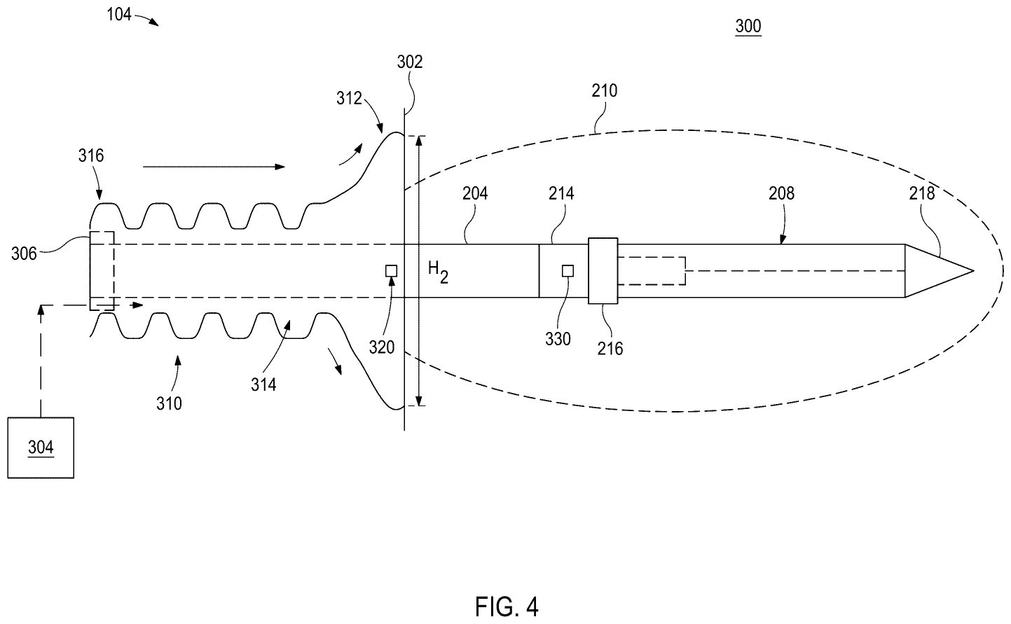

In the illustrated embodiment, the temperature regulator 310 is situated around the cannula 204 of the energy delivery device 104. In at least one embodiment, the temperature regulator 310 may be slidable relative thereto, such as by the user manually moving the temperature regulator 310 along the length of the cannula 204. The temperature regulator 310 may be comprised of a flexible material that allows a distal end 312 to splay (spread) outwardly along the skin 302 as the temperature regulator 310 is moved in a distal direction toward the skin 302. Specifically, in a relaxed state, as shown in FIG. 3, the distal end 312 of the temperature regulator 310 is in contact with a first area (with a first height H1) of the patent's skin 302. As the temperature regulator 310 is slid (moved) distally and into increased contact with the skin 302, as shown in FIG. 4, the distal end 312 may be configured to spread radially outward along the skin 302 such that the distal end 312 contacts an increased second area of the skin (with a second height H2) that is greater than the first area of the patient's skin 302. Consequently, the temperature regulator 310 may be able to provide additional cooling capabilities, as will be described in more detail below.

In some embodiments, the temperature regulator 310 comprises a flexible bellows. For example, the temperature regulator 310 may be made of a flexible elastomer, such as silicone, a flexible rubber, or “thubber” (e.g., a flexible, thermally conductive rubber). In other embodiments, the temperature regulator 310 may be made of a flexible polymer, such as polyurethane or neoprene. In other embodiments, the temperature regulator 310 may be made of an elastomeric, biocompatible material. In some embodiments, the temperature regulator 310 includes an insulative portion that a user can grasp to prevent the user's hand from being subjected to the reduced temperature of the temperature regulator 310, as described in more detail below.

In various embodiments, the temperature regulator 310 includes a cooling channel 314 that is fluidically coupled to a coolant supply 304. In some embodiments, the coolant source 107 comprises the cooling supply 304. In some embodiments, the coolant supply 304 may form part of the temperature adjustment system 118 (FIG. 1), and may be configured to store and provide a cooling fluid or “coolant fluid” to the temperature regulator 310. In at least one embodiment, the coolant fluid provided to the temperature regulator 310 may be the same as the coolant fluid provided to the handle 202 (FIG. 2) and/or the stick region 214.

The temperature regulator 310 is to receive the coolant fluid from the coolant supply 304 to control (i.e., reduce) the temperature of the temperature regulator 310. After circulating through the temperature regulator 310, the coolant fluid may be directed out of the temperature regulator 310, such as back toward the temperature adjustment system 118 (FIG. 1) or toward a coolant sink. In some embodiments, the cooling channel 314 may be spiral wound within the temperature regulator 310 to increase the surface area in which heat transfer to the coolant fluid can occur. In some embodiments, the cooling channel 314 extends from a proximal end 316 of the temperature regulator 310 to the distal end 312 and then back toward the proximal end 316 in a generally U-shaped path. In some embodiments, the temperature regulator 310 may include or provide a plurality of discrete cooling channels 314. In some embodiments, the cooling channel 314 may comprise an inbound conduit that fluidly coupled to the coolant supply 304 for receiving the coolant fluid, and an outbound conduit fluidly coupled to a coolant sink (not shown) for receiving used coolant fluid after passing through the cooling channel 314.

During example use of the energy delivery device 104, a user can, at any time, actuate the coolant supply 304 to provide coolant fluid to the temperature regulator 310. For example, as discussed above, as the energy delivery device 104 delivers energy to the patient 300, an ablation zone 210 is formed which may overlap the patient's skin 302. The user can monitor the temperature of the skin 302 and determine if the temperature thereof is becoming excessive, and thus, additional cooling is needed. Temperature sensors can be positioned at various points along the probe cannula 204, such as a temperature sensor 320 located at or near the patient's skin 302. In at least one embodiment, the temperature sensor 320 may be mounted to the temperature regulator 310 at the distal end 312 where the temperature regulator 310 contacts the skin 302. This may prove advantageous in providing real-time temperature data at or near the skin 302.

The temperature sensor 320 can be communicatively coupled (either wired or wirelessly) to the control system 102 (FIG. 1) to provide real-time temperature measurements to the control system 102. In such embodiments, the GUI 120 (FIG. 1) may display the temperature measurements obtained by the temperature sensor 320 for the user to monitor in real-time. The control system 102 may include a memory that stores a threshold temperature corresponding to a temperature in which skin damage is expected to occur, and the processor 114 (FIG. 1) may be programmed and otherwise configured to compare the real-time temperature measurements with the threshold temperature. Based on the real-time temperature approaching, reaching, and/or exceeding the temperature threshold, the control system 102 can provide an alert informing the user that a corrective action is required, such as actuating (initiating operation of) the temperature regulator 310. In some embodiments, the alert comprises a visual alert displayed on the GUI 120. In other embodiments, the alert may comprise an audible alert derived from an audio module (e.g., a speaker) included in the control system 102. In yet other embodiments, or in addition to the foregoing, the handle 202 (FIG. 2) may be configured to provide tactile feedback (e.g., vibration, etc.), thus alerting the user with tactile feedback.

In some embodiments, the control system 102 (FIG. 1) automatically actuates the coolant supply to provide coolant fluid to the temperature regulator 310 when the threshold temperature is being approached, reached, and/or exceeded. In some embodiments, the control system 102 may be configured to adjust the rate of coolant fluid flow to the temperature regulator 310 based on the measured temperature of the skin 302. In one embodiment, the control system 102 provides a first rate of coolant fluid flow as the threshold temperature is being approached, a second rate of coolant fluid flow greater than the first rate when the threshold temperature is reached, and a third rate of coolant fluid flow greater than the second rate when the threshold temperature is exceeded. In some embodiments, the rate of coolant fluid flow to the temperature regulator 310 is a function of the measured temperature of the skin 302. In some embodiments, the control system 102 monitors the temperature of the skin 302 using the temperature sensor 320. In some embodiments, the control system 102 monitors the temperature of the skin 302 using a thermocouple coupled to the probe cannula 204. In other embodiments, the system 100 further comprises a skin temperature sensor to measure the temperature of the skin 302 and the control system 102 monitors the temperature of the skin 302 by receiving temperature measurements from the skin temperature sensor.

In some embodiments, a user can provide an input to the control system 102 (FIG. 1) when a user desires to actuate (initiate operation of) the temperature regulator 310. In one embodiment, the user can provide an input to the GUI 120 (FIG. 1), causing the processor 114 (FIG. 1) to actuate the temperature adjustment system 118 (FIG. 1) to provide coolant fluid to the cooling channel 314 of the temperature regulator 310. As shown in FIG. 3, the temperature regulator 310 can be slid (laterally moved) along the cannula 204 such that the distal end 312 abuts the patient's skin 302. In such a position (i.e., a relaxed position), the coolant fluid enhanced temperature regulator 310 can absorb heat received at the skin 302, reducing the chances of the skin 302 becoming damaged.

In some instances, the cooling provided by the temperature regulator 310 merely abutting the skin 302, as shown in FIG. 3, may not be sufficient to reduce the temperature of the skin 302 to an acceptable level, such as below the threshold temperature. With the temperature regulator 310 in the relaxed state, as shown in FIG. 3, the user and/or control system 102 can monitor the temperature of the skin 302, via the temperature sensor 320, to determine if additional cooling is needed. If the user and/or control system 102 (FIG. 1) determine that additional cooling is needed, the temperature regulator 310 can be moved further along the axial length of the cannula 204, causing the distal end 312 to engage the skin 302 more forcefully and expand radially outward to cover more surface area of the skin 302, as shown in FIG. 4. The increased surface area contact with the skin increases the heat transfer from the skin 302 to the temperature regulator 310, and thus, further reduces the chance of the patient's skin 302 becoming damaged.

Referring to FIG. 4, in some embodiments, the energy delivery device 104 may further include a lock 306 operable to help maintain the temperature regulator 310 in a desired position, such as in the compressed state against the patient's skin 302. With the temperature regulator 310 pressed against the patient's skin 302, the user may be able to actuate (e.g., lock) the lock 306 and thereby help retain the temperature regulator 310 compressed against the patient's skin 302. In some embodiments, the lock 306 is operable to position the temperature regulator 310 along the length of the cannula 214 to provide a variable degree of skin contact between the temperature regulator 310 and the skin 302. With the lock 306 locked, the user may be able to remove their hand from the temperature regulator 310 during the ablation procedure and the temperature regulator 310 will remain in the current position.

In some embodiments, the lock 306 may comprise an awning ring that is slidable along the cannula 214 and that can be secured at any location along the cannula 214 with a fastener, such as a screw (e.g., a set screw), for example. Once secured, the user can release the temperature regular 310, causing the proximal end 316 to spring backwards and abut the lock 306, securing the temperature regulator 310 in place. In some embodiments, the lock 306 comprises a Tuohy Borst adapter that is slidable along the cannula 214 and actuatable to engage the cannula 204 at a desired position thereof.

In some aspects, the temperature regulator 310 is considered a variable rate temperature regulator in that, in one state (FIG. 3), the temperature regulator receives (absorbs) thermal energy from the skin 302 at a first rate, and in another state (FIG. 4), the temperature regulator receives (absorbs) thermal energy from the skin 302 at a second rate greater than the first rate. Accordingly, a user can slide (position) the temperature regulator 310 into more or less contact with the skin 302 depending on a desired rate of cooling to the skin 302. While only two states of the thermal regulator 310 are shown and described, it should be noted that the temperature regulator can be moved into more than two states to absorb thermal energy from the skin 302 at a plurality of rates. For example, the temperature regulator 310 could be moved into a third state in between the first state (FIG. 3) and the second state (FIG. 4) to absorb thermal energy from the skin at a third rate that is greater than the first rate, but less than the second rate. Furthermore, the temperature regulator can absorb a variable amount of thermal energy from the skin according to a flow rate of coolant fluid provided thereto from the coolant supply 304. Accordingly, a rate of thermal absorption via the thermal regulator 310 can be dependent upon a rate of coolant fluid flow provided thereto and/or an amount of surface area that is in contact with the skin 302. In some aspects, the temperature regulator 310 is considered a heat sink.

In some embodiments, to control the temperature of the skin 302, the control system 102 (FIG. 1) may be configured to control a rate of coolant fluid flow to both the stick region 214 and the temperature regulator 310. In such embodiments, rather than merely providing coolant fluid flow to the temperature regulator 310, the control system 102 further adjusts the coolant fluid flow to the stick region 214. A second temperature sensor 330 may be included in the stick region 214 and operable to measure a real-time temperature of the tissue surrounding the stick region 214. The second temperature sensor 330 may communicate with the control system 102 to provide real-time temperature measurements. In such embodiments, the control system 102 can retrieve, from a memory, a threshold cooling temperature and compare the real-time temperature to the threshold cooling temperature to determine if the temperature of the stick region 214 can be reduced any further without damaging the tissue surround the stick region 214. In some embodiments, the threshold temperature corresponds to a temperature in which skin damage due to excessive cooling is expected to occur Based on a determination that the temperature at the stick region 214 can be further reduced without damaging the skin 302, the control system 102, in combination with providing coolant fluid flow to the temperature regulator 310, can adjust the amount of coolant fluid provided to the stick region 214, thereby further increasing thermal absorption to prevent damage to the skin 302.

In some embodiments, a user may be able to selectively control a rate of coolant fluid flow to both the stick region 214 and the temperature regulator 310 by providing an input to the GUI 120 (FIG. 1), which may be configured to display information such that the user can determine how to control the temperature of the skin 320. In some embodiments, the coolant channels to the stick region 214 and the temperature regulator 310 can include flow sensors operable to sense the flow rate of coolant fluid to the same. In such embodiments, the GUI 120 can display the measured flow rates, the temperature of the skin 320, and the temperature at the stick region 214. Based on the displayed information, the user can provide an input to the GUI 120 to adjust the coolant fluid flow to the temperature regulator 310 and/or the stick region 214.

A typical ablation procedure with an energy delivery device 104 typically includes two portions. The first portion, as described above, is an ablation period in which the energy delivery device 104 delivers microwave energy to ablate tissue within a patient. During this period, the temperature regulator 310 can be used to control the temperature of the patient's skin 302 to prevent, or at least substantially reduce, the patient's skin 302 from being damaged.

The second portion is a cauterization period or process in which energy is provided to the antenna 208 as the cannula 204 is slowly withdrawn from the patient. The cauterization period is performed such that seed tumor cells are destroyed along the exit path of the cannula 204 as the energy delivery device 104 is withdrawn from the patient. During this cauterization period, the antenna 208 is pulled progressively closer to the skin 302 as the cannula 204 is withdrawn, thereby increasing the chances of the skin 302 overheating and becoming damaged. During the period, in some embodiments, the user can grasp and hold the temperature regulator 310 against the patient's skin 302 as the cannula 204 is withdrawn through the temperature regulator 310, further reducing the chances of the patient's skin 302 burning during use of the energy delivery device 104. In other embodiments, however, the temperature regulator 310 may be secured to the skin 302 during withdrawal of the cannula 204, such as with an adhesive that sticks the material of the temperature regulator 310 to the skin 302.

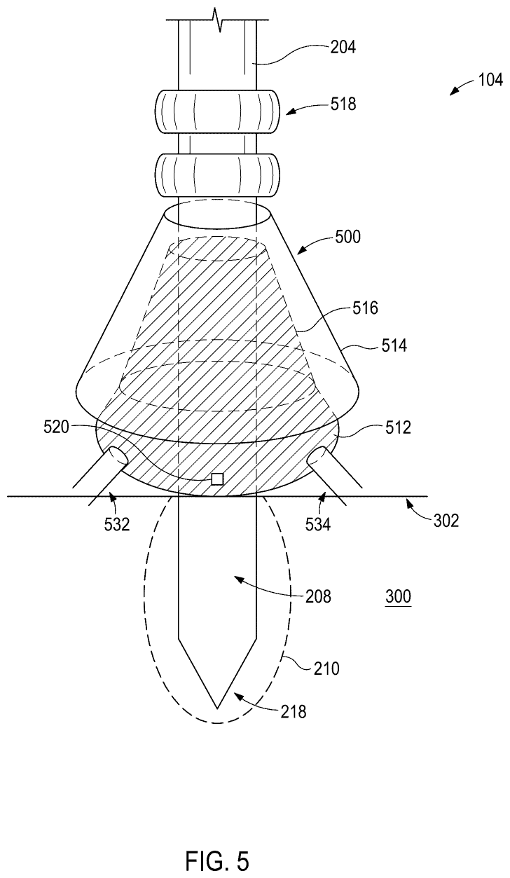

FIG. 5 is an enlarged view of another example of the energy delivery device 104, according to one or more additional embodiments of the present disclosure. Similar to the embodiment shown in FIGS. 3 and 4, the energy delivery device 104 is depicted as percutaneously inserted into the patient 300 with the antenna 208 positioned near the patient's skin 302. Moreover, the energy delivery device 104 may include a temperature regulator 510 that, similar to the temperature regulator 310 of FIGS. 3 and 4, functions to control the temperature of the patient's skin 302 to prevent damage thereto.

As illustrated, the temperature regulator 510 includes a flexible regulator portion 512, a first cone 514 surrounding the flexible regulator portion 512, a second cone 516 positioned within the first cone 514, and a lock 518. In some embodiments, the first and second cones 514, 516 are rigid cones.

In some embodiments, the first cone 514 and the second cone 516 are fixed to one another such that they are collectively movable along the cannula 204. In other embodiments, the first and second cones 514, 516 are separately movable along the cannula 204. In some embodiments, the flexible regulator portion 512 and the first and second cones 514, 516 are all coupled together such that they are collectively movable along the cannula 204. In other embodiments, however, the flexible regulator portion 512 and the first and second cones 514, 516 are separately movable along the cannula 204. In some embodiments, the temperature regulator 510 only includes one of the first or second cones 514, 516.

The flexible regulator portion 512 may be made of any of the flexible materials mentioned herein with respect to the temperature regulator 310 of FIGS. 3 and 4.

The lock 518 functions to engage the cannula 204 and (at least) the first cone 514 to prevent the temperature regulator 510 from moving proximally along the cannula 204 once fixed in place. In some embodiments, the lock 518 is coupled to the first cone 514 such that they are collectively movable along the cannula 204. In other embodiments, the lock 518 and the first cone 514 are separately movable along the cannula 204. The lock 518 may comprise a Tuohy Borst adapter, but could alternatively comprise an awning ring with a screw (e.g., a set screw).

The flexible regulator portion 512 may be similar in some aspects to the temperature regulator 310 of FIGS. 3 and 4. In some embodiments, for example, the flexible regulator portion 512 includes a coolant inlet 532 and a coolant outlet 534. The coolant inlet 532 is configured to convey coolant fluid into the flexible regulator portion 512 to control (i.e., reduce) the temperature thereof. The coolant inlet 532 may be fluidically coupled to a coolant supply to receive coolant fluid therefrom. In example operation, the coolant fluid may fill the flexible regulator portion 512 and thereby causing the flexible regulator portion 512 to expand or “balloon” up. The flexible regulator portion 512 may be configured to expand until filling the interior of the second cone 516. The coolant fluid may then be discharged from the flexible regulator portion 512 via the coolant outlet 534 and directed to a coolant sink.

In some embodiments, the coolant inlet 532 may enter through a first side of the flexible regulator portion 512 and the coolant outlet 534 may exit through a second side of the flexible regulator portion 512 opposite the first side, and otherwise on portions of the flexible regulator portion 512 that protrude from the frustoconical shape of the cones 514, 516. In other embodiments, the inlet and coolant outlets 532, 534 may be located on the same side of the flexible regulator portion 512. In yet other embodiments, and similar to the temperature regulator 310 of FIGS. 3 and 4, the inlet and coolant outlets 532, 534 may run alongside, or parallel, to the cannula 204 such that coolant fluid is supplied to and removed from the flexible regulator portion 512 alongside (in parallel with) the cannula 204.

During use of the energy delivery device 104, a user can, at any time, actuate the coolant supply to provide coolant fluid to the temperature regulator 510, and more specifically, to the flexible regulator portion 512. As discussed above, as the energy delivery device 104 delivers energy to the patient 300, an ablation zone 210 is formed which may overlap the patient's skin 302. The user and/or the control system 102 can monitor the temperature of the skin 302, determine if the temperature thereof is becoming excessive, such as via temperature sensors positioned along the cannula 204, and determine if additional cooling is needed.

When it is determined that additional cooling is needed for the skin 302, the user can distally (longitudinally) slide (translate) the temperature regulator 510 toward the patient's skin 302. In some embodiments, the flexible regulator portion 512 is moved distally to abut the patient's skin 302, the second cone 516 is moved distally to engage the flexible regulator portion 512, the first cone 514 is moved distally to encompass the second code, and the lock 518 is moved distally to engage the proximal end of the first cone 514. Once the user is satisfied with the position of the temperature regulator 510 relative to the patient's skin 302, the user can actuate (engage) the lock 518, preventing the temperature regulator 510 from moving proximally along the cannula 204.

In the alternative, the temperature regulator 510 can be positioned as discussed above at the outset of the ablation process, rather than when it is determined that additional cooling is required. In such instances, when it is determined that additional cooling is required for the skin 302, the control system 102 (FIG. 1) and/or the user can actuate (initiate flow of) the coolant supply without first needing to bring the temperature regulator 510 into an appropriate position relative to the skin 302.

Once it has been determined that additional cooling is required for the skin 302, the user and/or the control system 102 (FIG. 1) can control the coolant supply to provide coolant fluid through the coolant inlet 532 to the flexible regulator portion 512, thereby causing the flexible regulator portion 512 to expand within the second cone 516 and absorb thermal energy from the skin 302.

As the flexible regulator portion 512 is provided with coolant fluid, the flexible regulator portion 512 expands within the second cone 516 and along the patient's skin 302. Notably, the second cone 516 is held in place as the flexible regulator portion 512 expands, due to the lock 518 engaging the proximal end of the first cone 514 and the first cone 514 engaging the second cone 516. Accordingly, the frustoconical shape of the second cone 516 limits how much the flexible regulator portion 512 can expand in the proximal direction and encourages the flexible regulator portion 512 to expand laterally (e.g., radially outward) along the patient's skin 302, increasing the area in which the temperature regulator portion 512 contacts the skin 302 to absorb heat therefrom.

In some instances, the cooling provided by the temperature regulator 510 may not be sufficient to reduce the temperature of the skin 302 to an acceptable level, such as below the threshold temperature. The user and/or the control system 102 (FIG. 1) can monitor the temperature of the skin 302 via one or more temperature sensors 520 to determine if additional cooling is needed. The temperature sensor(s) 520 may be mounted to any or all of the first cone 514, the second cone 516, the flexible regulator portion 512, and the cannula 204.

If the user and/or control system 102 determines that additional cooling is needed, the user and/or the control system (FIG. 1) can increase the flow rate of coolant fluid through the coolant inlet 532, causing the flexible regulator portion 512 to expand radially outward and cover more surface area of the patient's skin 302, thus increasing the rate of thermal absorption from the skin 302 to the flexible regulator portion 512. In some embodiments, expansion of the flexible regulator portion 512 is limited to the diameter of the first cone 514. In other embodiments, expansion of the flexible regulator portion is limited to the diameter of the second cone 514.

Operation and use of the foregoing temperature regulators 310, 510 decreases the chances of patient's skin burning while using an energy delivery device 104. The temperature regulators 310, 510 are particularly useful in situations where an ablation procedure is to occur near the patient's skin. For example, ablation procedures in the shin region are almost exclusively near the patient's skin given the size of a typical shin and the relative position of the shin bone near the skin.

### Embodiments Disclosed Herein Include

A. An energy delivery device comprising a cannula extendable through skin of a patient, an antenna extending from the cannula and operable to deliver energy to tissue of the patient, and a temperature regulator movable along a length of the cannula and operable to control a temperature of the skin as the antenna provides energy to the tissue.

B. A system comprising an energy delivery device, a processor, and a memory. The energy delivery device includes a cannula extendable through skin of a patient, an antenna extending from the cannula and operable to deliver energy to tissue of the patient, a temperature regulator positioned on the cannula and fluidly communicable with a coolant supply, and a temperature sensor to measure a temperature of the skin. The memory stores computer readable instructions that, when executed by the processor, cause the processor to receive, from the temperature sensor, a measured temperature of the skin and control, from the coolant supply, a flow of coolant fluid to the temperature regulator based on the measured temperature of the skin.

C. A system comprising an energy delivery device and a control system including a user interface, a processor, and a memory. The energy delivery device includes a cannula extending through skin of a patient, an antenna extending from the cannula to deliver energy to tissue of a patient, a temperature regulator positioned on the cannula and fluidly communicable with a coolant supply, and a temperature sensor to measure a temperature of the skin. The control system is in communication with the temperature sensor. The control system is configured to display, on the user interface, the measured temperature of the skin, receive, on the user interface, a user input, and control, from the coolant supply, a flow of coolant fluid to the temperature regulator based on the user input.

Each of embodiments A, B, and C may have one or more of the following additional elements in any combination: Element 1: wherein the temperature regulator is movable between a first state in which the temperature regulator contacts a first area of the skin and a second state in which the temperature regulator contacts a second area of the skin greater than the first area. Element 2: wherein the temperature regulator is comprised of an elastomer. Element 3: wherein the temperature regulator comprises a bellows. Element 4: further comprising a lock to prevent proximal movement of the temperature regulator along the cannula and away from the skin. Element 5: wherein the temperature regulator comprises a flexible regulator portion and a cone arranged proximal to the flexible regulator portion, wherein the cone is to limit expansion of the flexible regulator portion. Element 6: wherein the temperature regulator is to receive coolant fluid from a coolant supply. Element 7: further comprising a stick region intermediate the cannula and the antenna, wherein the stick region and the temperature regulator are to co-operatively control the temperature of the skin based on the antenna providing energy to the tissue. Element 8: wherein the processor is to control the flow of coolant fluid to the temperature regulator based on a comparison of the measured temperature of the skin and a threshold temperature stored within the memory. Element 9: wherein the energy delivery device further comprises a stick region intermediate the cannula and the antenna, wherein the stick region is fluidly communicable with the coolant supply, and the processor is to control a flow of coolant fluid to the stick region based on the measured temperature of the skin. Element 10: wherein the temperature sensor is a first temperature sensor and the energy delivery device further includes a second temperature sensor to measure a temperature of the tissue at the stick region, wherein the processor is to receive, from the second temperature sensor, a measured temperature of the tissue at the stick region, and wherein the processor is to control a flow of coolant fluid to the stick region further based on the measured temperature of the tissue at the stick region. Element 11: wherein the temperature regulator is movable between a first state in which the temperature regulator contacts a first area of the skin and a second state in which the temperature regulator contacts a second area of the skin greater than the first area. Element 12: wherein the temperature regulator is comprised of an elastomer. Element 13: wherein the temperature regulator comprises a flexible regulator portion and a cone proximal to the flexible regulator portion to limit expansion of the flexible regulator portion. Element 14: wherein the energy delivery device further comprises a stick region intermediate the cannula and the antenna, wherein the stick region is fluidly communicable with the coolant supply, and the control system is to control a flow of coolant fluid to the stick region based on the user input. Element 16: wherein the temperature regulator is movable between a first state in which the temperature regulator contacts a first area of the skin and a second state in which the temperature regulator contacts a second area of the skin, wherein the second area is greater than the first area. Element 17: wherein the temperature regulator is comprised of an elastomer. Element 18: wherein the temperature regulator comprises a flexible regulator portion and a cone proximal to the flexible regulator portion to limit expansion of the flexible regulator portion.

By way of non-limiting example, exemplary combinations applicable to A, B, and C include: Element 1 with Element 2; Element 1 with Element 3; Element 1 with Element 4; Element 4 with Element 5; Element 6 with Element 7; Element 8 with Element 9; Element 9 with Element 10; Element 11 with Element 12; Element 12 with Element 13; Element 14 with Element 15; Element 15 with Element 16; Element 16 with Element 17; and Element 17 with Element 18 . . .

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. An energy delivery device, comprising:
a cannula extendable through skin of a patient;
an antenna extending from the cannula and operable to deliver energy to tissue of the patient; and
a temperature regulator movable along a length of the cannula and operable to control a temperature of the skin as the antenna provides energy to the tissue.

2. The energy delivery device of claim 1, wherein the temperature regulator is movable between:
a first state in which the temperature regulator contacts a first area of the skin; and
a second state in which the temperature regulator contacts a second area of the skin greater than the first area.

3. The energy delivery device of claim 1, wherein the temperature regulator is comprised of an elastomer.

4. The energy delivery device of claim 1, wherein the temperature regulator comprises a bellows.

5. The energy delivery device of claim 1, further comprising a lock to prevent proximal movement of the temperature regulator along the cannula and away from the skin.

6. The energy delivery device of claim 1, wherein the temperature regulator comprises:
a flexible regulator portion; and
a cone arranged proximal to the flexible regulator portion, wherein the cone is to limit expansion of the flexible regulator portion.

7. The energy delivery device of claim 1, wherein the temperature regulator is to receive coolant fluid from a coolant supply.

8. The energy delivery device of claim 1, further comprising a stick region intermediate the cannula and the antenna, wherein the stick region and the temperature regulator are to co-operatively control the temperature of the skin based on the antenna providing energy to the tissue.

9. A system, comprising:
an energy delivery device, comprising:
a cannula extendable through skin of a patient;
an antenna extending from the cannula and operable to deliver energy to tissue of the patient;
a temperature regulator positioned on the cannula and fluidly communicable with a coolant supply; and
a temperature sensor to measure a temperature of the skin; and
a processor and a memory storing computer readable instructions that, when executed by the processor, cause the processor to:
receive, from the temperature sensor, a measured temperature of the skin; and
control, from the coolant supply, a flow of coolant fluid to the temperature regulator based on the measured temperature of the skin.

10. The system of claim 9, wherein the processor is to control the flow of coolant fluid to the temperature regulator based on a comparison of the measured temperature of the skin and a threshold temperature stored within the memory.

11. The system of claim 9, wherein the energy delivery device further comprises a stick region intermediate the cannula and the antenna, wherein the stick region is fluidly communicable with the coolant supply, and the processor is to control a flow of coolant fluid to the stick region based on the measured temperature of the skin.

12. The system of claim 11, wherein the temperature sensor is a first temperature sensor and the energy delivery device further includes a second temperature sensor to measure a temperature of the tissue at the stick region, wherein the processor is to receive, from the second temperature sensor, a measured temperature of the tissue at the stick region, and wherein the processor is to control a flow of coolant fluid to the stick region further based on the measured temperature of the tissue at the stick region.

13. The system of claim 9, wherein the temperature regulator is movable between:
a first state in which the temperature regulator contacts a first area of the skin; and
a second state in which the temperature regulator contacts a second area of the skin greater than the first area.

14. The system of claim 9, wherein the temperature regulator is comprised of an elastomer.

15. The system of claim 9, wherein the temperature regulator comprises:
a flexible regulator portion; and
a cone proximal to the flexible regulator portion to limit expansion of the flexible regulator portion.

16. A system, comprising:
an energy delivery device, comprising:
a cannula extendable through skin of a patient;
an antenna extending from the cannula to deliver energy to tissue of a patient;
a temperature regulator positioned on the cannula and fluidly communicable with a coolant supply; and
a temperature sensor to measure a temperature of the skin; and
a control system in communication with the temperature sensor and including a user interface, a processor, and a memory, the control system being configured to:
display, on the user interface, the measured temperature of the skin;
receive, on the user interface, a user input; and
control, from the coolant supply, a flow of coolant fluid to the temperature regulator based on the user input.

17. The system of claim 16, wherein the energy delivery device further comprises a stick region intermediate the cannula and the antenna, wherein the stick region is fluidly communicable with the coolant supply, and the control system is to control a flow of coolant fluid to the stick region based on the user input.

18. The system of claim 16, wherein the temperature regulator is movable between:
a first state in which the temperature regulator contacts a first area of the skin; and
a second state in which the temperature regulator contacts a second area of the skin, wherein the second area is greater than the first area.

19. The system of claim 16, wherein the temperature regulator is comprised of an elastomer.

20. The system of claim 16, wherein the temperature regulator comprises:
a flexible regulator portion; and
a cone proximal to the flexible regulator portion to limit expansion of the flexible regulator portion.
